(19) United States
(12) Patent Application Publication
(10) Pub. No.: US 2026/0218317 A1
(43) Pub. Date: Jul. 30, 2026

(54) NUCLEIC ACID PROBES AND THEIR USES FOR THE DETECTION OF PREVOTELLA BIVIA

(71) Applicants: THE UAB RESEARCH FOUNDATION, Birmingham, AL (US); UNIVERSITY OF MINHO, Braga (PT)

(72) Inventors: Christina Muzny, Birmingham, AL (US); Nuno Miguel Dias Cerca, Braga (PT); Carina Manuela Fernandes Almeida, Braga (PT); Lucia Filipa Guimaraes Vieira de Sousa, Vieira do Minho (PT)

(73) Assignees: THE UAB RESEARCH FOUNDATION, Birmingham, AL (US); UNIVERSITY OF MINHO, Braga (PT)

(21) Appl. No.: 18/877,166

(22) PCT Filed: Jun. 27, 2023
(86) PCT No.: PCT/US2023/069174

§ 371 Date: Dec. 19, 2024

Related U.S. Application Data
(60) Provisional application No. 63/355,887, filed on Jun. 27, 2022.

(51) Int. Cl. C12Q 1/689; C12Q 1/6841

(52) U.S. Cl. CPC C12Q 1/689; C12Q 1/6841; C12Q 2600/158

(57) ABSTRACT

Provided herein are compositions and methods for detecting bacteria associated with bacterial vaginosis.

### PRIOR RELATED APPLICATION

This application is a U.S. National Phase of international application no. PCT/US2023/069174, filed on Jun. 27, 2023 entitled “NUCLEIC ACID PROBES AND THEIR USES FOR THE DETECTION OF PREVOTELLA BIVIA” which claims the benefit of and priority to U.S. Provisional Application No. 63/355,887, filed on Jun. 27, 2022, which is hereby incorporated by reference in their entirety.

### STATEMENT REGARDING FEDERALLY FUNDED RESEARCH

The invention was made with government support under AI146065 awarded by the National Institutes of Health. The government has certain rights in the invention

### REFERENCE TO A SEQUENCE LISTING SUBMITTED AS XML VIA EFS-WEB

The instant application contains a Sequence Listing which has been filed electronically in .xml format and is hereby incorporated by reference in its entirety. Said .xml copy, created on Jun. 27, 2023, is named 035979_1391561 seqlist.xml and is 15 kilobytes in size.

### BACKGROUND

Bacterial vaginosis (BV) remains the most common vaginal infection worldwide. The diagnosis of BV is usually performed using Amsel's criteria or the Nugent score. Currently, there are only a few FDA-approved nucleic acid amplification tests for detecting BV. However, these methods present limitations and can be used only for symptomatic women. Therefore, new compositions and methods for identification of BV-associated bacteria are necessary.

### SUMMARY

Provided herein are nucleic acid sequences for the detection of one or more bacteria associated with bacterial vaginosis. For example, provided herein is a nucleic acid probe comprising a nucleic acid sequence having at least 80% identity to SEQ ID NO: 1 (ATTAAACGTCCATGTCC), wherein the nucleic acid sequence is linked to a detectable moiety. In some embodiments, the detectable moiety is a fluorophore. In some embodiments, the nucleic acid sequence is a peptide nucleic acid (PNA) sequence. In some embodiments, the nucleic acid sequence specifically hybridizes to a Prevotella bivia nucleic acid.

Also provided is a method for detecting Prevotella bivia in a biological sample from a subject, comprising: (a) contacting the biological sample with any of the nucleic acid probes comprising a nucleic acid sequence having at least 80% identity to SEQ ID NO: 1; and (b) detecting the presence of a complex between the nucleic acid probe and a Prevotella bivia nucleic acid in the biological sample.

Also provided is a method of detecting one or more bacteria selected from the group consisting of Prevotella bivia (P. bivia), F. vaginae, and Gardnerella, in a biological sample from a subject, the method comprising: (a) contacting the biological sample with: (i) a first nucleic acid probe comprising a nucleic acid sequence having at least 80% identity to SEQ ID NO: 1; and/or (ii) a second nucleic acid probe comprising SEQ ID NO: 2 (CAGCATTACCACCCG), and/or (iii) a third nucleic acid probe comprising SEQ ID NO: 3 (CGATGTGCGACTAAA); and (b) detecting the presence of a complex between the nucleic acid probe comprising SEQ ID NO: 1 and a Prevotella bivia nucleic acid, a complex between the nucleic acid probe comprising SEQ ID NO: 2 and a Gardnerella nucleic acid; and/or a complex between the nucleic acid probe comprising SEQ ID NO: 3 and a F. vaginae nucleic acid.

In some embodiments, the sample is contacted with the first nucleic acid probe comprising a nucleic acid sequence having at least 80% identity to SEQ ID NO: 1. In some embodiments, the sample is contacted with the first nucleic acid probe comprising a nucleic acid sequence having at least 80% identity to SEQ ID NO: 1; and the second nucleic acid probe comprising SEQ ID NO: 2. In some embodiments, the sample is contacted with the first nucleic acid probe comprising a nucleic acid sequence having at least 80% identity to SEQ ID NO: 1; and the nucleic acid probe comprising SEQ ID NO: 3. In some embodiments, the sample is contacted with the first nucleic acid probe comprising a nucleic acid sequence having at least 80% identity to SEQ ID NO: 1; the second nucleic acid probe comprising SEQ ID NO: 2; and the third nucleic acid probe comprising SEQ ID NO: 3.

In some embodiments, the first nucleic acid probe is linked to a first detectable moiety. In some embodiments, the second nucleic acid probe is linked to a second detectable moiety. In some embodiments, the third nucleic acid probe is linked to a third detectable moiety. In some embodiments, each of the first, second and third detectable moieties is a different detectable moiety. In some embodiments, the detectable moiety is a fluorophore. In some embodiments, the first, second, and/or third nucleic acid probe is a PNA probe.

In some embodiments, the biological sample comprises cells, tissues or fluid obtained from a patient suspected of having bacterial vaginosis. In some embodiments, the biological sample comprises cells, tissues or fluid from a vaginal swab. In some embodiments, the one or more bacteria are detected using fluorescent in situ hybridization (FISH). In some methods, the detection of one or more complexes indicates the subject has bacterial vaginosis.

Also provided is a kit comprising: (i) a nucleic acid probe comprising a nucleic acid sequence having at least 80% identity to SEQ ID NO: 1, (ii) a nucleic acid probe comprising a nucleic acid sequence having at least 80% identity to SEQ ID NO: 2 (CAGCATTACCACCCG), and (iii) a nucleic acid probe comprising a nucleic acid sequence having at least 80% identity to SEQ ID NO: 3 (CGATGTGCGACTAAA). IN some kits, the nucleic acid sequences are PNA sequences.

### BRIEF DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows fluorescence microscopy results of P. bivia PNA probe hybridization. The images show examples of DAPI staining (DAPI filter) and different hybridizations of P. bivia PNA probe (FITC filter) for the strain of P. bivia ATCC 29303 (good hybridization), G. vaginalis ATCC 14018 (absence of hybridization), and F. vaginae ATCC BAA-55 (absence of hybridization). The images were acquired with a magnification of 400×.

FIG. 2 is a calibration curve of PbivPNA1454 probe efficiency in biofilm, determined by the correlation between cell counts with DAPI staining and the PNA probe. Each point represents average of counts from three independent experiments and error bars represent standard deviation.

FIGS. 3A and 3B show confocal laser scanning microscopy images of dual and triple-species biofilms analyzed by PNA-FISH. The images, in FIG. 3A, show the hybridization of the PNA probes PbivPNA1454 (green), Gard162 (red), and FvagPNA651 (cyan) in dual-species biofilms of G. vaginalis (Gv), P. bivia (Pb) and F. vaginae (Fv). The images were acquired with a magnification of 100×. FIG. 3B shows images of a triple-species biofilm, acquired using two different magnifications (100× and 400×). Scale bars represent 10 m.

### DETAILED DESCRIPTION

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

The vaginal microbiome is highly colonized by diverse microbial species living in close vicinity and often promoting mutual association with the host that promotes the maintenance of a healthy environment. The most common vaginal dysbiosis among women of reproductive age is bacterial vaginosis (BV) (Chen et al. “The Female Vaginal Microbiome in Health and Bacterial Vaginosis,” Front. Cell. Infect. Microbiol. 11, 631972 (2021)). BV is normally associated with an imbalance in the vaginal microbiota, characterized by a decrease of lactic acid-producing bacteria, normally Lactobacillus species, and an overgrowth of diverse anaerobic bacteria, including Gardnerella, Fannyhessea (for example, Fannyhessea vaginae), and Prevotella (for example, Prevotella bivia) species. The most common symptoms associated with BV are described as a vaginal discharge, an unpleasant odor, an increase of vaginal pH, and the presence of clue cells. Besides the physical discomfort and psychological and social distress, BV has also been related to some other serious complications, such as preterm birth, pelvic inflammatory disease, and acquisition of sexually transmitted diseases.

The diagnosis of BV is commonly performed by a clinical assessment using Amsel's criteria or by the Nugent score. The Amsel criteria rely on the observation and inspection of the common symptoms associated with the infection, namely the presence of a vaginal discharge, vaginal pH higher than 4.5, the release of a fishy smell on the addition of a 10% potassium hydroxide to a drop of vaginal discharge and the presence of clues cells on microscopic analysis. Three out of the four criteria used in the method must be present to diagnose a positive case of BV. The Nugent score is considered the best method for diagnosis of BV and it includes the classification and quantification of the microorganisms present on vaginal wet smears. The smears are classified on a 0-10 scale and the resultant score indicates the presence of BV if it is equal to or greater than seven. However, these two methods present significant limitations such as, for example, the variability interobserver, which depends on the skills and experience of the technician. As mentioned above, FDA tests are only approved for symptomatic women. Further, none of the tests include detection of Prevotella bivia, which the present application identifies as being associated with bacterial vaginosis.

Provided herein are peptide nucleic acid (PNA) probes targeting Prevotella bivia, a BV-associated bacteria, as well as methods, for example, a multiplex approach for detection of Gardnerella spp., P. bivia and Fannyhessea vaginae. It is understood that Fannyhessea vaginae is also known as Atopobium vaginae and these designations an be used interchangeably. As described herein, the P. bivia PNA probes provided herein specifically detected the target species, i.e., P. bivia, and a multiplex approach detected the presence of the three species in multi-species BV biofilms.

### Nucleic Acid Sequences and Probes

Provided herein are nucleic acid sequences for the detection of P. bivia in a biological sample. In some embodiments, the nucleic acid sequence comprises, consists of, or consists essentially of a nucleic acid sequence having at least 80% identity to SEQ ID NO: 1 (ATTAAACGTCCATGTCC). Any of the nucleic acid sequences disclosed herein can be used in combination with other nucleic acid sequences to detect P. bivia and one or more non-P. bivia bacteria associated with BV, for example, Gardnerella spp and/or Fannyhessea vaginae (F. vaginae). In some embodiments, a nucleic acid sequence that comprises, consists of, or consists essentially of a nucleic acid sequence having at least 80% identity to SEQ ID NO: 2 (CAGCATTACCACCCG) can be used to detect Gardnerella spp. In some embodiments, a nucleic acid sequence comprising, consisting of, or consisting essentially of a nucleic acid sequence having at least 80% identity to SEQ ID NO: 3 (CGATGTGCGACTAAA) can be used to detect F. vaginae.

As used throughout, the term nucleic acid or nucleotide refers to deoxyribonucleic acids (DNA) or ribonucleic acids (RNA) and polymers thereof in either single- or double-stranded form. It is understood that when a DNA is described, its corresponding RNA is also described, wherein thymidine is represented as uridine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like. The complement or complementary sequence of any nucleic acid sequence described herein is also provided.

The term identity or substantial identity as used in the context of a nucleotide sequence or nucleic acid sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

some embodiments, the nucleic acid sequence set forth herein are locked nucleic acids (LNA) or LNA analogs. In an LNA, the 2′-hydroxyl group is linked to the 4′ carbon atom of the sugar ring thereby forming a 2′-C,4′-C-oxymethylene linkage, and thereby forming a bicyclic sugar moiety. The linkage can be a methylene (—CH2-) group bridging the 2′ oxygen atom and the 4′ carbon atom wherein n is 1 or 2 (Singh et al., “LNA (locked nucleic acids): synthesis and high-affinity nucleic acid recognition,” Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic oligonucleotides containing LNAs have been described (Wahlestedt et al., Proc. Nati. Acad. Sci. U.S.A., 2000, 97, 5633-5638). The synthesis and preparation of the LNA adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in International Application Publication Nos. WO98/39352 and WO99/14226, both of which are hereby incorporated by reference in their entirety. Exemplary LNA analogs are described in U.S. Pat. Nos. 7,399,845 and 7,569,686, both of which are hereby incorporated by reference in their entirety.

In some embodiments, the nucleic acid sequences set forth herein are peptide nucleic acids (PNA). Whereas DNA and RNA have a deoxyribose and ribose sugar backbone, respectively, a PNA's backbone displays 2-aminoethyl glycine linkages in place of the regular phosphodiester backbone of DNA, and the nucleotides bases are attached to this backbone at the amino-nitrogens through methylene carbonyl linkages. By convention, PNAs are depicted like peptides, with the N-terminus at the left (or at the top) position and the c-terminus at the right (or at the bottom) position. PNAs hybridise to complementary DNA or RNA sequences in a sequence-dependent manner, following the Watson-Crick hydrogen bonding scheme. See, for example, Egholm et al., “PNA hybridizes to complementary oligonucleotides obeying the Watson-Crick hydrogen-bonding rules”. Nature. 365 (6446): 566-8 (1993); Shakeel and Ali, “Peptide nucleic acid (PNA) a review,” Journal of Chemical Technology and Biotechnology 81(6): 892-899 (2006); and Pellestor and Paulasova, “The peptide nucleic acids (PNAs), powerful tools for molecular genetics and cytogenetics,” 12: 694-700 (2004)). Since the backbone of PNA contains no charged phosphate groups, the binding between PNA/DNA strands is stronger than between DNA/DNA strands due to the lack of electrostatic repulsion. A representative structure for a PNA and exemplary binding to DNA is shown below. The amide bond characteristic of a PNA is boxed in.

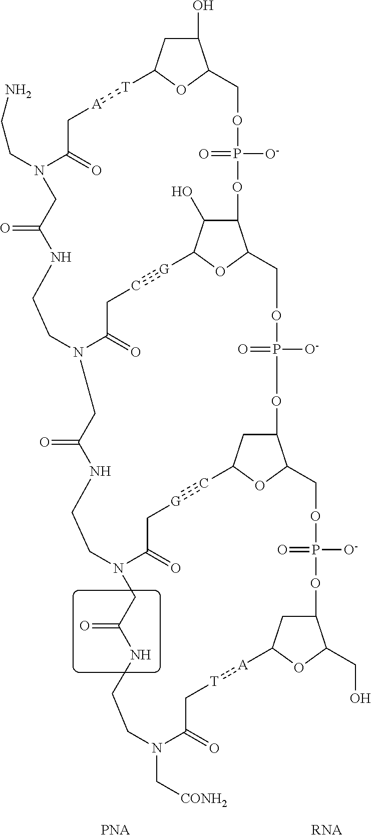

Any of the nucleic acid sequences set forth herein can be used as probes in detection methods to detect one or more bacteria associated with BV. In some embodiments, the probe is labeled with a detectable moiety. The term detectable moiety refers to a molecule or material that can produce a detectable (such as visually, electronically or otherwise) signal that indicates the presence (i.e. qualitative analysis) and/or concentration (i.e. quantitative analysis) of the label in a sample. In some embodiments, the nucleic acid sequences provided herein and the nucleic acid sequences linked to a detectable moiety are non-naturally occurring molecules and can be readily synthesized using methods known in the art.

The detectable moiety can be a fluorophore, which when excited by exposure to a particular stimulus such as a defined wavelength of light, emits light for example, at a different wavelength. Examples of fluorophores that can be used in the probes disclosed herein, include but are not limited to, Alexa Fluor 488, 4-acetamido-4′-isothiocyanatostilbene-2,2′disulfonic acid; acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanosine; 4′, 6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC), -6-carboxy-fluorescein (HEX), and TET (Tetramethyl fluorescein); fluorescamine; IR144; IR1446; Malachite Green isothiocyanate; 4-methylumbelliferone; ortho cresolphthalein; nitrotyrosine; pararosanilin; Phenol Red; B-phycoerythrin; o-phthaldialdehyde; pyrene and derivatives such as pyrene, pyrene butyrate and succinimidyl 1-pyrene butyrate; Reactive Red 4 (CIBACRON™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, N,N,N′,N′-tetramethyl-6-carboxyrhodamine (TAMRA), tetramethyl rhodamine, and tetramethyl rhodamine isothiocyanate (TRITC); sulforhodamine B; sulforhodamine 101 and sulfonyl chloride derivative of sulforhodamine 101 (Texas Red); riboflavin; rosolic acid and terbium chelate derivatives; LightCycler Red 640; Cy5.5; and Cy56-carboxyfluorescein; boron dipyrromethene difluoride (BODIPY); acridine; stilbene; 6-carboxy-X-rhodamine (ROX); Texas Red; Cy3; Cy5, VIC® (Applied Biosystems); LC Red 640; LC Red 705; and Yakima yellow amongst others.

The nucleic acid sequences disclosed herein are perfectly complementary or substantially complementary (e.g., having 1-4 mismatches) to a sequence of a target nucleic acid present within a bacteria associated with BV such that, if said target nucleic acid is present in a biological sample, at least a portion of said target nucleic acid hybridizes to at least a portion of said probe nucleic acid to form a double-stranded portion of nucleic acid. In some embodiments, the entire probe hybridizes to the target nucleic acid in a sample.

As used herein, the term hybridize or specifically hybridize refers to a process where two complementary nucleic acid strands anneal to each other under appropriately stringent conditions. Hybridizations are typically conducted with probe-length nucleic acid molecules. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y. In some embodiments, hybridization of one or more probes described herein is performed at temperatures of about 50° C. to about 63° C., (for example, from about 50° C. to about 60° C., or about 55° C. to about 60° C.) for about 60 to about 90 minutes. In some embodiments, the temperature is about 50° C., 51° C., 52° C., 53° C., 54° C., 55° C., 56° C., 57° C., 58° C., 59° C., 60° C., 61° C., 62° C., or 63° C. In some embodiments, the incubation time is about 60 to about 90 minutes, about 60 to 80 minutes, about 60 to 70 minutes or about 60 to about 65 minutes.

Kits comprising any of the nucleic acid sequences or probes described herein and reagents for determining the presence or absence of one or more bacteria associated with BV are also provided. In some embodiments, the kit comprises (a) one or more nucleic acid sequences or probes for the detection of a bacteria associated with BV, for example, a nucleic acid sequence or probe comprising SEQ ID NO: 1, a nucleic acid sequence or probe SEQ ID NO: 2 and/or a nucleic acid sequence or probe SEQ ID NO:3. In some embodiments, the kit comprises a probe comprising SEQ ID NO: 1, a probe comprising SEQ ID NO: 2 and a probe comprising SEQ ID NO:3 for the detection of P. bivia, Gardnerella spp and F. vaginae, respectively. In some embodiments the one or more nucleic acid sequences are PNA sequence or probes. Optionally, each probe can further comprise or be linked to a detectable moiety. In some embodiments, each probe is linked to a different detectable moiety, for example, a different fluorophore, for the simultaneous detection of two or more bacterial species in a sample. Optionally, the kit further comprises reagents and buffers for use in fluorescent in situ hybridization (FISH) assays.

### Methods for Detection of BV-associated Bacteria

Also provided are methods for detecting BV-associated bacteria, e.g., in a biological sample. For example, provided herein is a method for detecting Prevotella bivia in a biological sample from a subject, comprising: (a) contacting the biological sample with any of the nucleic acid probes comprising a nucleic acid sequence having at least 80% identity to SEQ ID NO: 1; and (b) detecting the presence of a complex between the nucleic acid probe and a Prevotella bivia nucleic acid in the biological sample. In some embodiments, the method further comprises contacting the biological sample with one or more probes that detect the presence of one or more non-P. bivia bacteria associated with BV. In some embodiments, the one or more bacteria comprise F. vaginae, and/or Gardnerella bacteria.

Also provided is a method of detecting one or more bacteria selected from the group consisting of Prevotella bivia, F. vaginae, and Gardnerella, in a biological sample from a subject, the method comprising: (a) contacting the biological sample with: (i) a first nucleic acid probe comprising a nucleic acid sequence having at least 80% identity to SEQ ID NO: 1; and/or (ii) a second nucleic acid probe comprising a nucleic acid sequence having at least 80% identity to SEQ ID NO: 2 (CAGCATTACCACCCG), and/or (iii) a third nucleic acid probe comprising a nucleic acid sequence having at least 80% identity to SEQ ID NO: 3 (CGATGTGCGACTAAA); and (b) detecting the presence of a complex between the nucleic acid probe comprising SEQ ID NO: 1 and a Prevotella bivia nucleic acid, a complex between the nucleic acid probe comprising SEQ ID NO: 2 and a Gardnerella nucleic acid; and/or a complex between the nucleic acid probe comprising SEQ ID NO: 3 and a F. vaginae nucleic acid.

In the methods provided herein the nucleic acid probe comprising SEQ ID NO: 1, SEQ ID NO: 2, or SEQ ID NO: 3 binds to or specifically hybridizes with a target nucleic acid in Prevotella bivia, F. vaginae, and Gardnerella, respectively, based on complementarity, to form a complex. It is understood that detection of a complex is used interchangeably with detecting hybridization. As used herein, the term specifically hybridizes means that under given hybridization conditions a probe detectably hybridizes substantially only to its target sequence(s) in a sample comprising the target sequence(s) (i.e., there is little or no detectable hybridization to non-targeted sequences).

In some embodiments, the sample is contacted with the first nucleic acid probe comprising a nucleic acid sequence having at least 80% identity to SEQ ID NO: 1. In some embodiments, the sample is contacted with the first nucleic acid probe comprising a nucleic acid sequence having at least 80% identity to SEQ ID NO: 1; and the second nucleic acid probe comprising SEQ ID NO: 2. In some embodiments, the sample is contacted with the first nucleic acid probe comprising a nucleic acid sequence having at least 80% identity to SEQ ID NO: 1; and the nucleic acid probe comprising SEQ ID NO: 3. In some embodiments, the sample is contacted with the first nucleic acid probe comprising a nucleic acid sequence having at least 80% identity to SEQ ID NO: 1; the second nucleic acid probe comprising SEQ ID NO: 2; and the third nucleic acid probe comprising SEQ ID NO: 3.

In some embodiments, the first nucleic acid probe is linked to a first detectable moiety. In some embodiments, the second nucleic acid probe is linked to a second detectable moiety. In some embodiments, the third nucleic acid probe is linked to a third detectable moiety. In some embodiments, each of the first, second and third detectable moieties is a different detectable moiety. In some embodiments, the detectable moiety is a fluorophore. Exemplary fluorophores are described above. In some embodiments, the first, second, and/or third nucleic acid probe is a PNA probe.

As used herein, by subject is meant a female subject or individual. Preferably, the subject is a mammal such as a primate, and, more preferably, a human. The female subject can be an adult subject or a pediatric subject. Pediatric subjects include subjects ranging in age from birth to eighteen years of age.

Any of the methods of detecting BV-associated bacteria described herein can be used to diagnose a subject with bacterial vaginosis. In some cases, the female subject is symptomatic for BV. In some cases, the female subject is asymptomatic for BV. As used herein the terms diagnose, diagnosis or diagnosing, refer to distinguishing or identifying a disease, syndrome or condition or distinguishing or identifying a person having a particular disease, syndrome or condition. In some embodiments of the invention, detection of one or more complexes (for example, a DNA probe-genomic DNA complex) in a biological sample, indicative of the presence of one or more BV-associated bacteria, is used to diagnose bacterial vaginosis in the subject. Any of the methods provided herein can include one or more controls, for example, a positive and/or a negative control. Any of the methods described herein can be used in combination with, for example, a subject's medical history, a pelvic examination, detection of clue cells, wet mount, and/or a vaginal pH test to diagnose bacterial vaginosis.

The methods and compositions of this invention may be used to detect nucleic acids associated with various bacteria using a biological sample obtained from a subject. The nucleic acid (DNA or RNA) may be isolated from the sample according to any methods well known to those of skill in the art. Biological samples can be obtained by standard procedures known to those of skill in the art. Once collected or obtained, samples can be used immediately or stored, under conditions appropriate for the type of biological sample, for later use.

In some cases, the sample is a clinical sample which is suspected to contain one or more bacteria associated with BV, for example, Prevotella bivia, F. vaginae, and/or Gardnerella. In some cases, the nucleic acids can be separated from proteins and sugars present in the original sample using methods known in the art.

Methods of obtaining test samples include, but are not limited to, swabs (e.g., vaginal swabs), biofilms, aspirations, tissue sections, swabs, drawing blood or other fluids, surgical or needle biopsies, and the like. The test sample can be obtained from an individual or patient. The sample can comprise cells, tissues or fluid obtained from a patient suspected of having bacterial vaginosis. The sample can be a cell-containing liquid or a tissue. Samples include, but are not limited to, cells from a vaginal swab, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples can also be processed, such as sectioning of tissues, fractionation, purification, or cellular organelle separation.

If necessary, the sample can be collected or concentrated by centrifugation and the like. In some embodiments, the cells of the sample can also be lysed, for example, by enzymatic treatment, heat, surfactants, ultrasonication, or a combination thereof. The lysis treatment is performed in order to obtain a sufficient amount of nucleic acid derived from the bacterial cells to perform hybridization assays. It is understood that any method or assay known or later developed that uses hybridization to detect the presence of one or more BV-associated bacteria described herein can be used. For example, these hybridization assays include, but are not limited to, blotting techniques, flow cytometry, polymerase chain reaction (PCR), DNA-DNA hybridization, and fluorescence in situ hybridization (FISH). In some cases, FISH flow cytometry is used.

In some embodiments, FISH is used to detect one or more BV-associated bacteria in a biological sample, as described in the Examples. Methods for sample preparation, (for example, bacterial cell preparation), and FISH analysis are known in the art. See, for example, Sousa et al. “A New PNA-FISH Probe Targeting Fannyhessea vaginae.” Front. Cell. Infect. Microbiol. 11, 1162 (2021); Stender “PNA FISH: an intelligent stain for rapid diagnosis of infectious diseases,” Expert Rev. Mol. Diagn. 3(5): 649-655 (2003); and Cerqueira et al. “PNA-FISH as a new diagnostic method for the determination of clarithromycin resistance of Helicobacter pylori,” BMC Microbiology 11: 101 (2011).

Any of the detection methods described herein can further comprise treating a subject for bacterial vaginosis. Treating or treatment of any disease or disorder refers to ameliorating a disease or disorder that exists in a subject. The term ameliorating refers to any therapeutically beneficial result in the treatment of a disease state, e.g., bacterial vaginosis, lessening in the severity or progression, delaying or preventing recurrence of the disease, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both.

A subject diagnosed with BV using any of the methods described herein can be treated with an effective amount of one or more of metronidazole (e.g., Flagyl or Metrogel-Vaginal), clindamycin (e.g. Cleocin or Clindesse), tinidazole (Tindamax) or secnidazole (Solosec). One or more rounds of treatment can be administered to the subject depending on the general health of the subject, the genetic disposition of the subject, diet, time of administration, rate of excretion, drug combination, age or size of the subject, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

### Additional Definitions

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

“About” is used to provide flexibility to a numerical range endpoint by providing that a given value may be “slightly above” or “slightly below” the endpoint without affecting the desired result.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/of” refers to and encompasses any and all possible combinations of one or more of the associated listed items, as well as the lack of combinations where interpreted in the alternative (“of”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Example I—PNA Probes and Multiplex Assay

### PNA Probe in Silico Design

A PNA probe specific for the detection of P. bivia was designed following the protocol of set forth in Machado, et al. “Fluorescence in situ Hybridization method using Peptide Nucleic Acid probes for rapid detection of Lactobacillus and Gardnerella spp.” BMC Microbiol. 13, 82 (2013); and Sousa et al. “A New PNA-FISH Probe Targeting Fannyhessea vaginae.” Front. Cell. Infect. Microbiol. 11, 1162 (2021)). A set of sequences from 16S and 23S rRNA collections was selected from the Arb-Silva database (Version 138.1) (https://www.arb-silva.de/search/) with lengths >1200 bp or >1600 bp, respectively, and quality scores >90. The P. bivia rRNA sequences available in this database, as well as sequences from closely related bacterial species, were then aligned using the Clustal Omega tool (https://www.ebi.ac.uk/Tools/msa/clustalo/). Conserved regions of the sequences were chosen as potential probes, based on perfect matches with sequences of interest and mismatches with sequences of non-interest. The theoretical sensitivity and specificity of the probes were determined, as described in Almeida, et al. “Rapid detection of urinary tract infections caused by Proteus spp. using PNA-FISH,” Eur. J. Clin. Microbiol. Infect. Dis. 32, 781-786 (2013), and the probes were tested using the TestProbe tool from Arb-Silva with no mismatches allowed. The chosen sequences were then classified based on the number of target strains/species, the position of mismatches in closely related strains/species, the percentage of GC content between 40% and 60%, melting temperature >50° C., Gibbs free energy between −13 kcal/mol and −20 kcal/mol, and theoretical values of sensitivity and specificity. The Gibbs free energy and the melting temperature were calculated (See, for example, Owczarzy et al. “Stability and mismatch discrimination of locked nucleic acid-DNA duplexes,” Biochemistry 50, 9352-9367 (2011). The sequence with the best theoretical results was selected for probe synthesis (Eurogentec, Seraing, Belgium) and the probe was linked to an Alexa Fluor molecule via a double 8-amino-3,6-dioxaoctanoic acid linker.

### Bacterial Growth Conditions

Several strains of P. bivia and other BVAB were used to determine the experimental sensitivity and specificity of the P. bivia probe. The bacteria were grown on Columbia Agar Base (Liofilchem, Roseto degli Abruzz, Italy) supplemented with 5% (v/v) of defibrinated horse blood (Oxoid, Basingstoke, UK), excluding S. sanguinegens that was grown on chocolate agar supplemented with 10% of inactivated horse serum (Biowest, Nuaillé, France). The plates were incubated at 37° C. and 10% CO₂ for 48 h. For anaerobic bacteria (i.e., Actinomyces urogenitalis, Aerococcus christensenii, Bifidobacterium bifidum, Campylobacter ureolyticus, F. vaginae, L. iners, Megasphaera micronuciformis, Mobiluncus curtisii, M. mulieris, Mycoplasma hominis, Peptostreptococcus anaerobius, Porphyromonas asaccharolytica, Prevotella spp., Propionibacterium acnes, S. sanguinegens and Veillonella parvula), the plates were maintained under anaerobic conditions using anaerobic gas-generating packs (AnaeroGen Atmosphere Generation system, Oxoid). Acinetobacter baumannii was grown at 30° C. for 24 h.

### PNA-FISH Procedure

For the PNA-FISH procedure, a bacterial suspension of each strain was prepared in 1× phosphate buffered saline (PBS) solution and with the optical density (OD) adjusted to 0.1, followed by a two-fold dilution. Twenty μL of the suspension was then spread on epoxy-coated microscope glass slides with ten wells (Thermo Fisher Scientific, Lenexa, KS) and left to dry at 37° C. After this time, the fixation and permeabilization step was performed using 10 μL of 100% (v/v) methanol (Thermo Fisher Scientific) for 15 min, followed by 20 μL of 4% (w/v) paraformaldehyde (Thermo Fisher Scientific) for 10 min, and 10 μL of 50% (v/v) ethanol (Thermo Fisher Scientific) for 15 min. Afterward, the slides were left to dry at room temperature. The hybridization step was then performed by adding 10 μL of hybridization solution¹² containing 200 nM of PNA probe to each well of the slides and covering the slides with a coverslip. The slides were then incubated in moist and opaque containers at different temperatures and times. Time and temperature optimizations for hybridization were performed using the strain P. bivia ATCC 29303. Temperatures between 53° C.-63° C. and times of 60-90 min were evaluated. The optimized time and temperature were used for sensitivity and specificity determination experiments. After this time, the slides were removed and immersed in washing solution and incubated again for an additional 30 min at the same temperature. After washing, the slides were allowed to air dry and protected from light until microscopic analysis.

### Microscopic Analysis

Microscopic analysis was performed using an Olympus BX51 epifluorescence microscope (Olympus, Lisbon, Portugal) equipped with the FITC filter (BP 470-490, FT500, LP 516 sensitive to the Alexa Fluor 488 molecule). The fluorescence signal of the probe was observed using the filter FITC and the other filters were used to discard any autofluorescence signal from the cells. A negative control was included in the experiments with no probe added to the hybridization solution. The images were acquired with a 40× objective (Numerical aperture: 0.75) and using the same time of exposure for target and non-target species.

### Experimental Sensitivity and Specificity Determination

For determination of the experimental sensitivity and specificity of the P. bivia PNA probe, sixteen different strains of P. bivia and forty-eight other species associated either with BV or vaginal microbiome were used, respectively. The procedure of PNA-FISH hybridization was performed and the values of sensitivity and specificity were calculated, as described in Almeida et al., The experiments were repeated with, at least two, independent assays for each species/strain evaluated.

Determination of P. bivia PNA Probe Efficiency in Biofilm

Single-species biofilms of P. bivia were performed on 24-wells plates (Orange Scientific, Braine L'Alleud, Belgium) for 24 h. Briefly an inoculum of P. bivia ATCC 29303 was prepared in New York City III (NYCIII) broth medium supplemented with 10% (v/v) of inactivated horse serum and incubated in anaerobic conditions for 24 h. After that, the inoculum concentration was adjusted to 10⁷ CFU/mL, and 1 mL dispensed on each well. The plate was incubated at 37° C. in anerobic conditions. After 24 h, the medium was removed and the biofilms were washed once with PBS. The biofilms were then resuspended on 1 mL of PBS and four serial dilutions were performed. Twenty microliters of each dilution were spread on epoxy microscope glass slides and set to dry. The fixation and hybridization procedures were performed as described above, using a temperature of 60° C. and 90 min for hybridization with the PbivPNA1454 probe. Before microscopic analysis, the cells were stained with DAPI at 2.5 μg/μL. For each dilution, 20 fields were randomly visualized, and the cells were counted per image with the DAPI staining and the PNA probe. The experiments were performed with three independent assays.

### Multiplex Approach for Quantification of Species in Triple-Species Biofilms

To optimize the PNA-FISH multiplex approach for BV research, the P. bivia probe was used with two other PNA probes: Gard162 (Machado et al.) with an Alexa Fluor 594, and FvagPNA651 (Sousa et al.) with an Alexa Fluor 633 (Eurogentec). Triple-species biofilms were grown in 24-well plates. For each experiment, an inoculum of each species of G. vaginalis ATCC 14018, F. vaginae ATCC BAA-55, and P. bivia ATCC 29303 was prepared in NYCIII supplemented with 10% (v/v) of inactivated horse serum and incubated at 37° C. and anaerobic conditions for 24 h. After 24 h, the bacterial concentration of the inoculums was determined by reading the optical density (OD) at 620 nm and adjusted to 10⁷ CFU/mL by a dilution in NYCIII broth. For each species, 111 μL of the adjusted suspension was combined on each plate well for a final volume of 1 mL. The biofilms were incubated for 24 h, at 37° C. in anaerobic conditions. After that time, the biofilms were washed once with PBS and then resuspended on 1 mL of PBS. Therefore, serial dilutions of triple-species biofilms were performed and 20 μL of each dilution was spread on epoxy glass slides and set to dry. The fixation and the hybridization steps were performed, using the 3 probes in the hybridization step, with the hybridization conditions of 60° C. and 90 min.

Using the fluorescence microscope, 20 images of each dilution were randomly taken using the filters appropriated for the observation of each probe. The cells from each species were counted and the composition of the triple-species biofilms was determined using the efficiency of each PNA probe. The experiments were performed with three independent assays.

### Results

A PNA probe was designed for the detection of P. bivia and an optimized a multiplex approach combining this probe with two additional PNA probes specific for Gardnerella spp. and F. vaginae as a new method for detection of these three common BVAB was developed. A total of sixteen 16S and ten 23S P. bivia sequences were retrieved from the Arb-Silva database (Version 138.1) and used for alignment in the probe development. The selected 23S rRNA sequences showed better results than the 16S rRNA sequences, with several conserved regions with potential to be used in the probe for P. bivia. Final probe selection was based on a greater number of perfect matches with P. bivia and a lower number of matches with non-target bacterial species. Theoretical sensitivity and specificity were determined by testing a total of 157,859 sequences from the 23S Arb-Silva REF collection, using the TestProbe tool, from which only ten sequences corresponded to P. bivia strains. The theoretical sensitivity was 100% and specificity was 99.9%, since the probe also targeted one out of four P. denticola sequences present in the database. The Gibbs free energy and the melting temperature were −17.92 kcal/mol and 56.9° C., respectively. This P. bivia probe was subsequently named PbivPNA1454: Alexa Fluor 488-OO-ATTAAACGTCCATGTCC (SEQ ID NO: 1). Alexa Fluor™ 488 is a bright, green-fluorescent dye with excitation suited for the 488 nm laser line. Optimizations in the temperature and incubation time were performed and the best signal-to-noise ratio was obtained at about 58° C. for about 90 min.

The results of hybridization were qualitatively classified based on a four-level scale (Machado et al.), including the absence of hybridization (−), poor (+), moderate (++), and good (+++) hybridization (Table 2). Despite showing distinct levels of hybridization, all the strains hybridized with the P. bivia PNA probe (Table 2), which indicates an experimental sensitivity of 100%, 95% confidence interval (CI) [79.4%, 100.0%]. None of the other related species showed a signal of hybridization with the probe (Table 1) indicating an experimental specificity of 100%, 95% CI [92.6%, 100.0%].

TABLE 1
Determination of analytical specificity of PbivPNA1454 probe. Results
of hybridization of P. bivia PNA probe with different species

| | | Hybridization |
|---|---|---|
| Species | Reference | results |
| Acinetobacter baumannii | CCUG 59798 | − |
| Actinomyces neuii | UM067 | − |
| Actinomyces urogenitalis | CCUG 44038 | − |
| Aerococcus christensenii | CCUG 28826 | −* |
| Bacillus firmus | UM034 | −* |
| Bifidobacterium bifidum | CCUG 59492 | − |
| Brevibacterium ravenspurgense | CCUG 42923 | − |
| Campylobacter ureolyticus | CCUG 44295 | − |
| Corynebacterium tuscaniense | UM137 | − |
| Enterococcus faecalis | UM035 | −* |
| Escherichia coli | UM056 | − |
| Fannyhessea vaginae | ATCC BAA-55 | −* |
| Gardnerella leopoldii | UM034 | −* |
| Gardnerella piotii | UM035 | −* |
| Gardnerella swidsinskii | UM094 | −* |
| Gardnerella vaginalis | ATCC 14018 | −* |
| Gemella haemolysans | UM034 | −* |
| Lactobacillus crispatus | EX533959VCO6 | − |
| Lactobacillus gasseri | ATCC 9857 | −* |
| Lactobacillus iners | ATCC 55195 | − |
| Lactobacillus rhamnosus | CECT 288 | − |
| Lactobacillus vaginalis | UM062 | − |
| Megasphaera micronuciformis | CCUG 45952T | − |
| Mobiluncus curtisii | ATCC 35241 | − |
| Mobiluncus mulieris | ATCC 35239 | −* |
| Mycoplasma hominis | UM054 | −* |
| Neisseria gonorrhoeae | CCUG 13281 | −* |
| Nosocomiicoccus ampullae | UM121 | − |
| Peptostreptococcus anaerobius | ATCC 27337 | − |
| Porphyromonas asaccharolytica | CCUG 7834T | − |
| Prevotella buccalis | CCUG 44127 | − |
| Prevotella copri | CCUG 58058T | − |
| Prevotella denticola | CCUG 29542T | − |
| Prevotella disiens | CCUG 59491 | − |
| Prevotella intermedia | CCUG 31410 | − |
| Prevotella melaninogenica | CCUG 65141 | −* |
| Prevotella nigrescens | CCUG 25289 | −* |
| Prevotella timonensis | CCUG 59487 | − |
| Propionibacterium acnes | UM034 | − |
| Shigella spp. | UM137 | − |
| Sneathia sanguinegens | CCUG 66076 | −* |
| Staphylococcus epidermidis | UM066 | − |
| Staphylococcus haemolyticus | UM066 | − |
| Staphylococcus hominis | UM224 | − |
| Staphylococcus saprophyticus | UM121 | − |
| Staphylococcus simulans | UM059 | − |
| Streptococcus agalactiae | UM035 | − |
| Veillonella parvula | CCUG 59474 | − |

Hybridization results were evaluated qualitatively according to the classification: (−) Absence of hybridization; (+) Poor hybridization; (++) Moderate hybridization; (+++) Good hybridization.
*These species showed some autofluorescence signal detected in the FITC filter.

TABLE 2
Determination of analytical sensitivity of PbivPNA1454
probe. Results of hybridization of P. bivia PNA
probe with different strains of P. bivia

| | | Hybridization |
|---|---|---|
| Strain | Reference | results |
| Prevotella bivia | ATCC 29303 | +++ |
| Prevotella bivia | CCUG 33360 | ++ |
| Prevotella bivia | CCUG 33961 | +++ |
| Prevotella bivia | CCUG 33962 | +++ |
| Prevotella bivia | CCUG 34043 | +++ |
| Prevotella bivia | CCUG 34044 | ++ |
| Prevotella bivia | CCUG 34045 | ++ |
| Prevotella bivia | CCUG 34046 | ++ |
| Prevotella bivia | CCUG 34047 | +++ |
| Prevotella bivia | CCUG 35221 | ++ |
| Prevotella bivia | CCUG 35880 | +++ |
| Prevotella bivia | CCUG 36740 | ++ |
| Prevotella bivia | CCUG 44195 | +++ |
| Prevotella bivia | CCUG 48913 | +++ |
| Prevotella bivia | CCUG 56865 | ++ |
| Prevotella bivia | CCUG 59496 | +++ |

Hybridization results were evaluated qualitatively according to the classification: (−) Absence of hybridization; (+) Poor hybridization; (++) Moderate hybridization; (+++) Good hybridization.

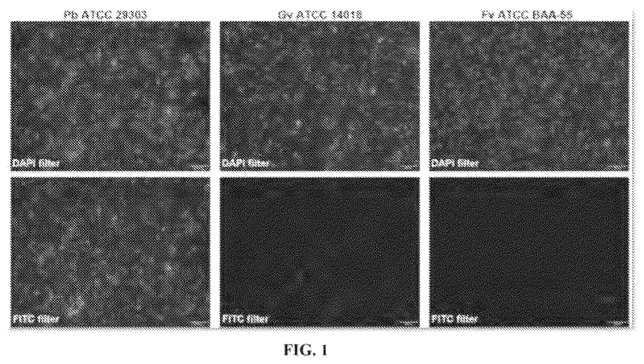

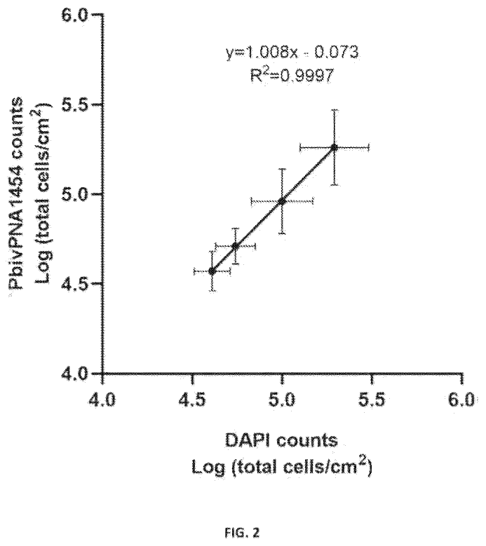

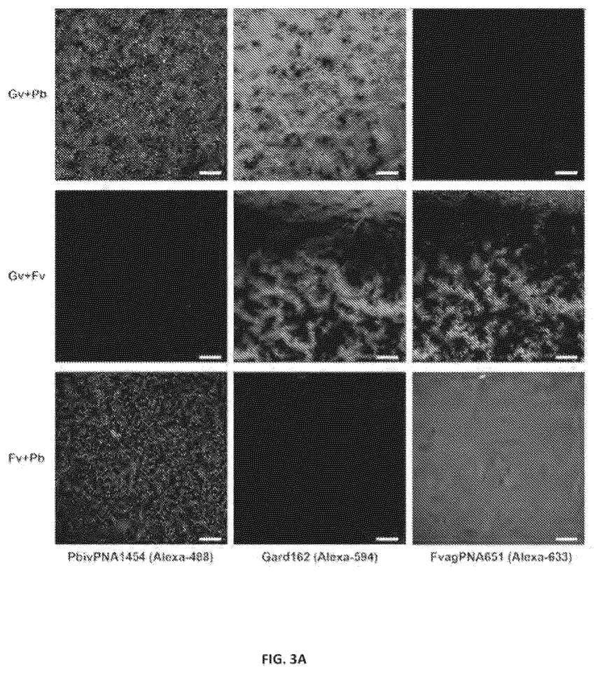

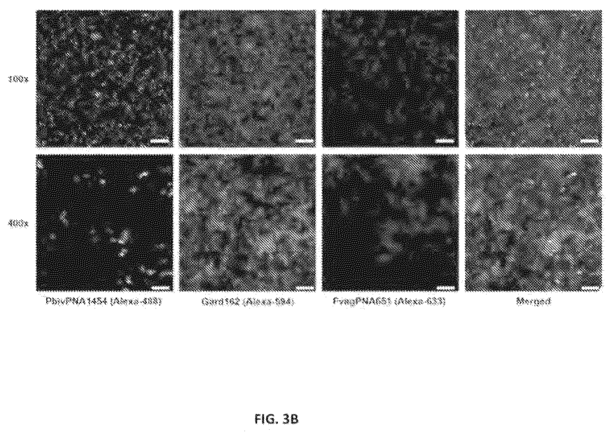

FIG. 1 shows examples of microscopic images from the hybridization of the P. bivia probe with G. vaginalis, P. bivia, and F. vaginae. A PNA multiplex protocol was developed, as described herein, for the combined detection of these three key BVAB. Keeping in mind that these bacterial species are frequently observed in women with BV, their detection could be an improvement for BV molecular diagnosis and research. For these experiments, dual and triple-species biofilms of G. vaginalis, F. vaginae, and P. bivia were analyzed using PNA probes. Since each probe had different optimal hybridization conditions, the best possible hybridization conditions for all the probes were determined. As such, based on optimization experiments for both the Gardnerella and F. vaginae probes and, considering the optimal conditions for each of the probes, exemplary conditions of about 60° C. and about 90 min were selected. However, hybridization conditions can include a temperature of about 50° C. to about 60° C., for about 60 to about 90 minutes. Before analysis of the triple-species biofilms, the efficiency of the P. bivia PNA probe was determined in biofilms at the hybridization conditions used in the multiplex protocol. Cell counts of P. bivia were performed using DAPI staining and the PNA probe, as shown in FIG. 2, resulting in an efficiency of 91.7%. FIG. 3A represents examples of hybridization of the three probes in dual-species biofilms. In all experiments, the probes were able to hybridize with the corresponding species, while not showing significant cross-hybridization with the other species. When analyzing triple-species biofilms at higher magnifications (FIG. 3B), it was also possible to observe the distribution of the three species in the biofilm. The probes were capable of differentiating between the spatial localization of each species. The composition of the triple-species biofilms was determined by cell counts using the specific PNA probes for G. vaginalis, F. vaginae, and P. bivia, which resulted in a biofilm composition (mean±SD) of 49.7% (±4.2%) of G. vaginalis, 45.7% (±3.4%) of F. vaginae, and 4.6% (±3.6%) of P. bivia. It was possible to distinguish the signal of the 3 species and the proportions found are in line with other studies that show that, in triple-species biofilms formed with the same species, P. bivia is usually outnumbered by other two species. In summary, a PNA approach for the detection of three key BVAB, including the development of a P. bivia PNA probe. Peptide Nucleic Acid Fluorescence in situ Hybridization (PNA-FISH) technique provides advantages compared to the conventional methods of microorganisms' identification, such as culture methods, since it is faster and more sensitive. PNA methods using the probes described herein, including PNA-FISH methods, are useful for detecting, diagnosing and treating BV.

### Multiplex Approach for Discrimination of Biofilm Species by CLSM

Dual and triple-species biofilms of G. vaginalis, F. vaginae and P. bivia were performed on 8-well chamber slides (Thermo Fisher Scientific™ Nunc™ Lab-Tek™). Each 24 h-inoculum, adjusted to 10⁷ CFU/mL, was then dispensed in the corresponding wells to perform the dual- and triple-species biofilm experiments, for a final volume of 400 μL in NYCIII. The chamber slides were then incubated at 37° C., in anaerobic conditions for 24 h. After 24 h, the biofilms were washed once using 0.9% (w/v) NaCl and set to dry at room temperature. After the fixation step, as described above, 30 μL of hybridization solution containing the 3 PNA probes, at a concentration of 200 nM, were dispensed on each well and covered with a coverslip. The PNA-FISH hybridization was conducted at 60° C. for 90 min followed by the washing step for 30 min. The biofilms were visualized using an Olympus™ Fluo-View FV1000 (Olympus) confocal laser scanning microscope (CLSM). The experiments were repeated twice.

### Statistical Analysis

Confidence intervals for experimental sensitivity and specificity were calculated based on the Clopper-Pearson method using the MedCalc software (Version 22.005, available at: https://www.medcalc.org/calc/diagnostic_test.php).

### Example II—BV and Biofilms

A notable feature of BV is the appearance of a multi-species biofilm on vaginal epithelial cells. While the biofilm likely contributes to high BV recurrence rates after therapy, it remains largely uncharacterized. It is known to contain abundant G. vaginalis, fewer Atopobium vaginae (also known as, and used interchangeably with Fannyhessea vaginae), and other various undefined bacterial species. G. vaginalis can displace Lactobacillus crispatus from HeLa cells and adhere in high concentrations to start biofilm formation. When certain BV-associated bacteria are incorporated into the G. vaginalis biofilm, G. vaginalis virulence genes are up-regulated. Therefore, it is possible that G. vaginalis initiates BV biofilm formation, but incident BV (iBV) requires incorporation of other key bacteria into the biofilm that alter the transcriptome of the polymicrobial consortium.

This is consistent with the finding that among women who have sex with women (WSW), the mean relative abundance of Prevotella bivia, G. vaginalis, and A. vaginae became sequentially higher prior to iBV. It is possible that a similar distribution of these bacterial species will increase prior to iBV in women who have sex with men (WSM). G. vaginalis and P. bivia exhibit a symbiotic relationship in vitro but whether P. bivia incorporates into the BV biofilm in vivo is unknown. A. vaginae rarely appears in the absence of G. vaginalis and is specific for BV. To determine the timing of the increase of these three organisms prior to iBV, frequent vaginal sampling (twice daily) is necessary. In addition, a better understanding of how these organisms interact in vivo and the molecular markers produced during BV biofilm formation is needed. These critical gaps in BV pathogenesis research can be addressed as described below.

### Changes in the Vaginal Microbiota Preceding iBV in a Longitudinal Study of WSM

It is hypothesized that prior to iBV, the mean relative abundance, and inferred absolute abundance, of G. vaginalis, P. bivia, and A. vaginae will become sequentially higher compared to women maintaining normal vaginal microbiota. Twice daily vaginal specimens will be obtained from 150 women with normal microbiota (Nugent score 0-3) and followed for iBV (Nugent score 7-10 on ≥4 consecutive specimens) over 60 days. 16S rRNA gene sequencing targeting V4 [and broad range 16S rRNA gene qPCR] will be done in women for the 14 days prior to iBV as well as age-comparable women maintaining normal microbiota. For women with normal microbiota, specimens chosen for sequencing will be based on menstrual cycle day, aligned with iBV subjects. In addition to traditional microbiome analysis (1) and inferred absolute bacterial abundance, SParse InversE Covariance Estimation for Ecological Association Inference and Weighted Gene Co-expression Network Analysis will be used to identify bacteria increasing in abundance prior to iBV.

Identify Molecular Markers Associated with iBV by Using RNA Sequencing to Analyze the Transcriptome of G. vaginalis, P. Bivia, and A. Vaginae

It is hypothesized that BV biofilm maturation depends on specific bacterial interactions. These interactions will be probed, first by RNA sequencing using in vitro models in a chemically defined medium simulating vaginal secretions (mGTS). Venn-diagram analysis will select the most commonly up-regulated genes shared in the in vitro assays. The in vivo relevance of potential molecular markers for the development of iBV will be assessed by qPCR, using vaginal specimens collected from women with iBV and negative controls discussed above.

These experiments will focus on three key BV-associated bacteria (G. vaginalis, P. bivia, and A. vaginae) and their role in iBV pathogenesis and includes both cultivation-independent studies and cultivation-dependent studies. These experiments could help identify the etiology of BV, thus improving BV diagnosis, treatment, and prevention. The studies described below were performed to support the hypothesis for these experiments.

In Silico and Experimental Evaluation of Primer Sets for Species Level Resolution of the Vaginal Microbiota using 16S rRNA Gene Sequencing

Prior studies have used different 16S primer sets, sequence databases, and parameters for sample and database clustering. This study assessed whether these methods could detect common BV-associated bacteria. An in silico analysis of 16S rRNA gene primer sets, targeting different hypervariable regions of the 16S rRNA gene was performed. 16S genes were sequenced in specimens from women with iBV using V1-V3, V3-V4, and V4 primer sets. Analysis relied on an extended Greengenes database including 16S gene sequences from vaginal bacteria not present in gg_13_5. Results were compared with the SILVA database. Using several database and sample clustering parameters, each primer set's ability to detect common BV-associated bacteria to the species level was determined. These methods were also compared to DADA2 for denoising and clustering of sequence reads. V4 sequence reads clustered at 99% identity, and using the 99% clustered, extended Greengenes database provided optimal species-level identification of common BV-associated bacteria. These data support sequencing of V4 and use of our 99% clustered, extended Greengenes database.

Identification of Key Vaginal Bacteria that Increase Prior to iBV

WSW with no Amsel criteria and a Nugent score of 0-3 were followed for iBV (Nugent score 7-10 on consecutive days). Participants self-collected vaginal swabs every day for 90 days. For WSW with iBV and WSW maintaining normal vaginal microbiota, 16S rRNA gene sequencing targeting V4 was performed on vaginal specimens for the 21 days prior to iBV. Sequencing data were processed using DADA2. Species-level taxonomy was assigned using PECAN with a vaginal-specific database. Longitudinal microbiome data were analyzed using the phyloseq library. Of 42 women enrolled, 31 completed the study, and 14/31 (45.2%) developed iBV. We sequenced 448 specimens (14 women with iBV, 8 maintaining normal vaginal microbiota). The mean relative abundance of P. bivia and G. vaginalis became significantly higher in women with iBV 4 days and 3 days before the day of iBV, respectively. The mean relative abundance of A. vaginae became significantly higher on the day of iBV onset. Because of the small sample size and once daily specimen collection, it could not be definitively determined whether the mean relative abundance of P. bivia increased before G. vaginalis did since the results were only 24-hours apart. Nevertheless, these data suggest that P. bivia, G. vaginalis, and A. vaginae play significant roles in iBV. Twice daily sampling will more precisely define the timing of the increase of these 3 bacteria prior to iBV.

Networks of Co-Abundant Vaginal Bacteria that Increase Prior to iBV

We analyzed vaginal specimens from the 14 women with iBV in the K23 study using SPIEC-EASI and WGCNA. A robust network of 3 bacteria (G. vaginalis, A. vaginae, and Aerococcus christensenii) was identified using SPIEC-EASI. This network was found to significantly increase prior to iBV using WGCNA (p<0.001). A. christensenii is particularly interesting since it has not been associated with iBV, although it is implicated in chorioamnionitis. Our traditional microbiome analysis did not focus on it. It is important to note that SPIEC-EASI identifies bacteria networks that increase and decrease together over sampling, whereas traditional analysis identified trends in relative abundance over time. This subtle differentiation means that P. bivia, which increased in relative abundance (along with G. vaginalis and A. vaginae) prior to iBV in our traditional microbiome analysis, did not form into a network with G. vaginalis and A. vaginae using SPIEC-EASI. This is because the timeframe of their increase was different. SPIEC-EASI and WGCNA will be applied to microbiome sequencing data. It is expected that associations between G. vaginalis and A. vaginae will be found, along with other bacteria that increase prior to iBV in WSM.

### Risk Factors for iBV Among WSM

WSM ages 18-45 with no Amsel criteria, Nugent score 0-3, and no evidence of STI were followed for iBV (Nugent score 7-10 on 3 consecutive days) with daily self-collected vaginal swabs for 60 days. Of 164 enrolled, 29 (17.7%) developed iBV at a median time of 21 (range 6-50) days. Women with iBV were significantly more likely to be older, African American, report a BV history, and have detectable G. vaginalis by qPCR at enrollment.]

### Study Design and Population

This prospective cohort study will recruit WSM from the Birmingham, AL metropolitan area using study flyers, word-of-mouth, advertising at local events (i.e. Magic City Classic, sporting events, sidewalk festivals, etc.), and local newspaper and radio advertisements. Eligible women presenting to the Jefferson County Health Department (JCDH) STD Clinic and the UAB Personal Health Clinic will also be recruited. These clinics serve a lower income, minority (>90% African American) population. In 2017 12,150 patients were seen at the JCDH STD clinic, 6,372 (52.4%) of which were women. In 2017, there were 1,440 female patient visits at the UAB Personal Health clinic. These recruitment methods were highly successful during our recent K23 study for which we screened 204 WSW over 3 years. It is expected that recruitment measures for this study will be even more successful as WSM represent a far larger proportion of the population of Birmingham, AL than WSW. In the event that these measures are not successful, study recruiters will be hired to advertise the study on foot throughout the local community.

Sex as a Biologic Variable. Since BV is highly prevalent in women and, with the exception of G. vaginalis-associated balanoposthitis, there is no known BV counterpart in men. Thus, the focus is on women.

Enrollment Numbers/Timeframe. Participant enrollment for changes in the vaginal microbiota preceding iBV in a longitudinal study of WSM is expected to occur over a 4-year period at UAB (Months 6-54). Based on power calculations (see Statistical Power), 150 women with normal vaginal microbiota who will collect twice daily vaginal specimens for 60 days must be enrolled. In a prior study at the JCDH STD clinic, approximately 40% of WSM had normal vaginal microbiota at baseline. It will be necessary to screen 400 WSM to enroll 160 with normal vaginal flora since it is expected that ~10 women will refuse to participate.

### Clinical Procedures

Study participation will proceed in 2 phases: screening and enrollment. Table 3 lists screening inclusion and exclusion criteria.

TABLE 2
Aim 1 Screening Inclusion and Exclusion Criteria

| Inclusion criteria | Exclusion criteria |
|---|---|
| Female sex | Use of oral or intra-vaginal antibiotics |
| | within 14 days |
| Age 18-45 years | HIV infection |
| History of sex with men | Pregnancy |
| Current male sexual partner | Current menses |
| | Sex with women |

Women meeting these criteria will be asked to self-collect one vaginal swab for determination of Amsel criteria and Nugent score while research staff will take a detailed sexual history and perform a urine pregnancy test. Non-pregnant women with no Amsel criteria, a Nugent score of 0-3 (determined by a research clinician and confirmed by a second reader in our lab), and no G. vaginalis morphotypes on vaginal Gram stain will be invited to enroll. Participants will complete a study questionnaire on socio-demographics, alcohol/tobacco/drug use, [antibiotic use], sexual history, douching history, and contraception use. A pelvic exam will be performed with a vaginal swab obtained for Trichomonas vaginalis, Chlamydia trachomatis, Neisseria gonorrhoeae nucleic acid amplification testing using the BD ProbeTec Qx CTQ/GCQ/TVQ assay and [Mycoplasma genitalium PCR testing using BioGX reagents (Birmingham, AL) on the BD Max platform. Women will complete a one-page daily diary (a yes/no checklist on their oral, vaginal, and anal sexual activities, [antibiotic use], sex toy use, partner gender and type, douching, and vaginal symptoms) for 60 days. They will be taught to self-collect vaginal specimens twice daily for 60 days, one of which will be used to prepare a Gram stain slide for Nugent scoring by study staff. The 3 swabs will be stored in specimen tubes for Aim 1-3 studies. [Vaginal specimen #1 will be used for microbiome and broad-range 16S rRNA gene qPCR sequencing in Aim 1. Vaginal specimen #2 will be used for PNA FISH experiments in Aims 2B and 2C. Vaginal specimen #3 will be used for qPCR in Aim 3B.] Participants will bring daily diaries, vaginal Gram stains, and vaginal specimen tubes on ice to the study site weekly for 60 days or until iBV (Nugent score of 7-10 on ≥4 consecutive specimens). Women testing positive for T. vaginalis, C. trachomatis, N. gonorrhoeae, [or M. genitalium] will be dropped since these STIs may alter the vaginal microbiota.

Vaginal Specimen Preservation for Microbiome [and Broad-Range 16S rRNA Gene qPCR] Sequencing, PNA FISH, and CSLM Assays

Participants will store vaginal specimen #'s 1 and 2 in a plastic bag at −20° C. (freezer), until delivery to the study site. Once the specimens arrive at our lab, Vaginal specimen #1 will be archived at −80° C. until shipped to LSUHSC for vaginal microbiome sequencing [and broad-range 16S rRNA gene qPCR]. An aliquot from Vaginal specimen #2 will immediately be transferred to an immunofluorescence slide and a smear will be prepared using standardized techniques. The slide will be fixed with 4% paraformaldehyde and stored at 4° C. until PNA FISH is performed. Paraformaldehyde, a cross-linking fixative agent compatible with fluorescent stains, creates covalent bonds between proteins that preserves bacterial cell structure, including the cell wall. This will ensure that the specimens on the slides remain intact prior to performing the appropriate studies.

Vaginal Specimen Preservation for RNA Extraction and qPCR

Participants will be instructed to store Vaginal specimen #3 in a plastic bag at −20° C. (freezer) until delivery to the study site. Up to 1 week later, the collection tubes, labeled “RNA” and preloaded with 250 μL of RNA-protect, will be achieved at −80° C. in the laboratory until they are shipped on dry ice from UAB to Minho University.

Vaginal Specimen Selection for Microbiome [and Broad-Range 16S rRNA Gene qPCR] Sequencing.

Vaginal specimen #1 will be classified per Nugent score. Women with a Nugent score of 7-10 on ≥4 consecutive specimens will be categorized as iBV. Age-comparable women with a Nugent score of 0-3 on twice-daily specimens for ≥85% of study days will be classified as maintaining normal vaginal microbiota. The remaining women will be classified as intermediate. For women with iBV, twice-daily specimens for the 14 days prior to iBV will be sequenced. Based on data obtained in our laboratory, it is hypothesized that the incubation period of BV is between 4-7 days. Thus, we have chosen to sequence the 14 days prior to iBV to provide an adequate timeframe to detect complex changes that occur prior to iBV. For women maintaining normal microbiota, twice-daily specimens for 14 days of sequencing will be chosen based on menstrual cycle day, aligned with women with iBV.

### DNA Extraction

LSUHSC will perform DNA extraction for selected vaginal specimens from women with iBV and women with normal vaginal microbiota using the QIAamp DNA Stool Mini Kit (QIAGEN, Germantown, MD), modified to include bead beating. Extracted DNA will be divided into two aliquots for 16S rRNA gene sequencing and qPCR measurements, both to be performed at LSUHSC.]

16S rRNA Gene Sequencing Methods

To prepare the sequencing library, two amplification steps will be performed using the AccuPrime Taq high fidelity DNA polymerase system (Thermo-Fisher/Invitrogen/Life Technologies, Carlsbad, CA). First, the 16S ribosomal DNA hypervariable region V4 will be amplified using 20 ng of genomic DNA and the gene-specific primers with Illumina adaptors: forward 5′ TCGTCGGCAGCGTCAGATGTGTATAAGAGACAG GTGCCAGCMGCCGCGGTAA (SEQ ID NO: 4) 3′; reverse 5′ GTCTCGTGGGCTCGGAGATGTGTATAAGAGACAG GGACTACHVGGGTWTCTAAT 3′ (SEQ ID NO: 5). Second, purified amplicon DNA from the last of 25 PCR cycles will be amplified for 8 cycles using the primers with different molecular barcodes: forward 5′ AATGATACGGCGACCACCGAGATCTACAC [i5] TCGTCGGCAGCGTC 3′ (SEQ ID NO: 6); reverse 5′ CAAGCAGAAGACGGCATACGAGAT [i7] GTCTCGTGGGCTCGG 3′ (SEQ ID NO: 7). The normalized and pooled libraries will be run with paired-end sequencing on an Illumina MiSeq (Illumina, San Diego, CA) using the 500 base pairs (bp) V2 sequencing kit (2×250 bp paired end reads).

### Bioinformatics Analysis of Vaginal Microbiome Sequence Data

Sequencing data will be analyzed using the DADA2 v1.8 software package in R (128). Briefly, sequence reads will be filtered and trimmed based on read quality profiles, selecting appropriate trimming parameters that maintain high quality base calls. Forward and reverse reads will be merged and chimeric reads will be removed. Taxonomic assignment will be performed using the RDP classifier (144) with our 99% clustered Greengenes database extended to include additional vaginal bacteria (108) (http://metagenomics.lsuhsc.edu/ExpandedGreengenes/). Secondary analysis will use the Phyloseq v1.24 (130) package in R to assess alpha and beta diversity and taxonomic summaries. Longitudinal microbiome analysis will be assessed via heatmaps produced through Phyloseq (1). SPIEC-EASI (42) followed by WGCNA (43) will be applied to the data to identify robust networks of co-expressed bacteria that increase significantly prior to iBV (106). These networks will be assessed for their association with time prior to iBV.

### Adjustment of 16S Relative Abundance Data to Inferred Absolute Abundance

A qPCR assay based on TaqMan chemistry will be used to measure copy number of 16S using the following primers that target the V3-V4 conserved region of the 16S rRNA: 16S338F 5′ ACTCCTRCGGGAGGCAGCAG 3′ (SEQ ID NO: 8),16S806R 5′ GGACTACCVGGGTATCTAAT 3′ (SEQ ID NO: 9), 16SPrb515R (Hydrolysis probe) 5′ 6-FAM-TKACCGCGGCTGCTGGCAC-TAMRA-BHQ1 3′ (SEQ ID NO: 10). These primers will be synthesized and high pressure liquid chromatography (HPLC) purified through Integrated DNA Technologies. Core reagents for the TaqMan Fast Advanced Master Mix will be ordered from Thermo Fisher Scientific. In a 20 μl reaction system, the final concentrations for both the forward and reverse primers will be added at 0.4 μM and probe concentration will be 0.2 μM. The reagent master will be filtered to minimize contamination using a Microcon YM-100 centrifugal filter unit (Millipore) at 2000 rpm for 35 minutes, 5000 rpm for 5 minutes, and 8000 rpm for 5 minutes. Reactions will start at 50° C. for 2 minutes on the BioRad CF96 real-time cycler followed by denaturation at 95° C. for 20 seconds. Reactions will then undergo 42 cycles of amplification with 95° C. melt for 15 seconds, 55° C. annealing for 39 seconds, and 72° C. extension for 30 seconds. Escherichia coli plasmid standards will be run for each reaction ranging from 107 to 10 gene copies; values will be reported as 16S rRNA gene copies/specimen to estimate the total bacterial load. Measurements for total bacterial load will be used to adjust relative abundance measures acquired from microbiome sequencing data. Additionally, HPLC primers will be ordered for targeted qPCR of G. vaginalis (Fw 5′-CACATTGGGACTGAGATACGG-3′ (SEQ ID NO: 11), Rv 5′-AGGTACACTCACCCGAAAGC-3′) (SEQ ID NO: 12), P. bivia (Fw 5′-CGCACAGTAAACGATGGATG-3′ (SEQ ID NO: 13), Rv 5′-ATGCAGCACCTTCACAGATG-3′) (SEQ ID NO: 14), and A. vaginae (Fw 5′-TATATCGCATGATGTATATGGG-3′ (SEQ ID NO: 15), Rv 5′-CATTTCACCGCTACACTTGG-3′) (SEQ ID NO: 16). Each qPCR assay will be performed in triplicate.]

### Statistical Power.

Table 4 illustrates standardized effect sizes that can be detected with ≥80% power using a two tailed Type I error rate of 0.05, assuming various autocorrelation (rho) and iBV event rates using a repeated measures study design with a sample size of 150 women and 28 vaginal specimens (2 specimens per day*14 days prior to iBV).

TABLE 4
Statistical Power for Various Standardized Effect Sizes

| | Sample Size = 150* | | | | | |
|---|---|---|---|---|---|---|
| IBV Event | | Normal | | Statistical Power | | |
| Rate | With IBV | Microbiota | Rho | 80% | 85% | 90% |
| 10% | 15 | 75 | 0.2 | 0.38 | 0.41 | 0.44 |
| 10% | 15 | 75 | 0.3 | 0.45 | 0.48 | 0.52 |
| 10% | 15 | 75 | 0.4 | 0.51 | 0.55 | 0.60 |
| 14% | 21 | 69 | 0.2 | 0.33 | 0.36 | 0.39 |
| 14% | 21 | 69 | 0.3 | 0.40 | 0.43 | 0.46 |
| 14% | 21 | 69 | 0.4 | 0.45 | 0.49 | 0.52 |
| 18% | 27 | 63 | 0.2 | 0.31 | [symbol] | 0.36 |
| 18% | 27 | 63 | 0.3 | 0.37 | 0.39 | 0.43 |
| 18% | 27 | 63 | 0.4 | 0.42 | 0.45 | 0.48 |

*The remaining participants will have a Nugent score of 4-6.

The standardized effect size is calculated by dividing the difference between the two group means (mean_{iBV}−mean_{normalvaginaimicrobiota}) by a common standard deviation. The autocorrelation will account for the fact that repeated observations for the same woman are not independent observations; non-independence in repeated measurements increases variation. As the autocorrelation increases (Table 4), larger effect sizes are required to achieve adequate statistical power. Higher iBV rates will permit detection of smaller differences. Table 4 clearly shows that across various autocorrelations and event rates, the study will have adequate power to detect small-medium standardized effect sizes. The circled table cell can be interpreted as follows: assuming an iBV rate of 18% (5) (27 women with iBV and 63 women with normal vaginal microbiota), a standard deviation (SD) of 10%, and an autocorrelation of 0.20, the study will have 85% power to detect a mean difference of 3.3% (0.33*10%) in the mean relative and absolute abundance of a given bacterial species between the two groups. Similarly, with a SD of 20%, a mean difference of 6.6% (0.33*20%) will be detected. Additional estimations (not shown) assumed that 14 vaginal specimens will be available instead of 28, since some women might not collect twice daily. With 14 vaginal specimens, the standardized effect size detected for the same table cell described above is 0.35; a similar difference of ~0.02 was observed for other cells. [If the final sample size for analysis is 10% smaller than expected (i.e. n=135, iBV=24, normal=57) due to STI infection at enrollment or loss-to follow-up, the effect size for the circled table cell will still be 0.35.] Power calculations were estimated using Power and Sample Size software, v14 (NCSS, Kaysville, UT).

### Statistical Analysis

Women with iBV will be compared to those maintaining normal vaginal microbiota with regards to various characteristics including the mean relative abundance [and inferred absolute abundance] of G. vaginalis, P. bivia, A. vaginae, and other common BV-associated bacteria. Continuous variables such as age will be compared using an unpaired t-test or Wilcoxon rank sum test, as appropriate. Socio-demographics, STI history, sexual behavior history, and contraception use at enrollment will be compared between groups using chi-squared or Fisher's exact tests. Longitudinal patterns in the sequencing data will then be examined using different approaches such as repeated measures ANOVA and linear mixed models which will account for missing data. We will compare the results using these approaches and will report the result for which the assumptions will be most reasonable. If the data demonstrate extreme skewness, we will use bootstrapping to conduct a non-parametric analysis; 10,000 bootstrapped samples will be drawn with replacement from women with iBV and women maintaining normal vaginal microbiota to create 95% confidence intervals (CIs) comparing the mean relative abundance difference between groups. This bootstrapping approach will select the available set of longitudinal values for each participant to maintain and account for the correlation structure among the repeated observations. Mean relative abundance differences between women with iBV and women maintaining normal vaginal flora, [after adjustment for antibiotic use and other characteristics,] will be calculated within each bootstrap for each sample. Bootstrap CIs will be estimated using a Bonferroni correction (conservative approach) adjusting the 0.05 Type I error rate for the available number of samples (i.e., 0.05/28=0.0017 when 28 samples are available). If the Bonferroni corrected bootstrap empirical CIs do not include zero, we will conclude that the groups differ significantly at that time point. Secondary analyses will examine the association of various socio-demographic characteristics, menses, douching, and sexual risk behaviors (collected in the enrollment questionnaire and the daily diaries) with iBV. Statistical significance will be set at 0.05 (two-tailed) except while examining individual samples when the Bonferroni correction will be applied, as stated above. Inferred absolute abundance of vaginal bacteria of interest, including G. vaginalis, P. bivia, and A. vaginae, will be obtained by multiplying each bacteria's relative abundance (measured by 16S rRNA gene sequencing) by their total bacterial load (measured by broad-range 16S rRNA gene qPCR). The relationship between the inferred absolute abundance and targeted qPCR absolute abundance of G. vaginalis, P. bivia, and A. vaginae will be examined by scatter plots and Pearson/Spearman correlation coefficients. Furthermore, agreement between the two measures will be examined by the Bland-Altman method for repeated measures. If agreement is high (i.e. 80% of the points within 2 standard deviations [SD]), the inferred absolute abundance of other vaginal bacteria will be used in our analyses as targeted qPCR will only be performed for G. vaginalis, P. bivia, and A. vaginae. If agreement is not high, we will instead use absolute abundance data of G. vaginalis, P. bivia, and A. vaginae from targeted qPCR in our analyses. All analyses will be conducted using SAS, v9.4 (Cary, NC).

### Expected Outcomes, Limitations, and Alternatives

Expected outcomes include: (1) The mean relative [and inferred absolute] abundance of G. vaginalis, P. bivia, and A. vaginae will sequentially increase (in that order) in WSM in the days leading up to iBV, compared to women maintaining normal vaginal microbiota. If BV is an STI, its pathogenesis should be the same whether women have sex with women or with men. (2) Networks of co-abundant bacteria which move together prior to iBV will be found; a subset of these bacteria will be the ones we propose to study in BV biofilm formation. (3) WGCNA analysis will reveal that networks involving biofilm-forming BV-associated bacteria will increase prior to iBV.

Determine the Contribution of P. bivia to the Multi-Species BV Biofilm In Vivo.

Significant in vitro and in vivo studies support the presence of G. vaginalis and A. vaginae in the multi-species biofilm that is a hallmark of BV development. However, based on prior research, many other bacterial species are known to be present in women with BV. Whether or not these other species are involved in the BV biofilm is unknown. To confirm the involvement of a given bacterial species in the BV biofilm, specific microscopy probes, such as PNA probes, must be developed. This aim is designed to determine the contribution of P. bivia to the multi-species BV biofilm in vivo. It is expected that P. bivia will incorporate into the BV biofilm after G. vaginalis and before A. vaginae.

While no in vivo studies have explored the incorporation of P. bivia in the BV biofilm, we have found that, like A. vaginae, P. bivia co-aggregates with G. vaginalis and can incorporate a multi-species BV biofilm in vitro. These data support the hypothesis that, along with G. vaginalis, P. bivia and A. vaginae play key roles in BV biofilm pathogenesis.

Further, PNA probes efficiently detect bacteria in biofilms, as their uncharged nature reduces interaction with matrix components improving diffusion within 3D structures. This demonstrated that PNA probes can be used to distinguish 3 or more bacterial species in biofilm communities.

Using the P. bivia PNA probes described herein, several outcomes are expected. The P. bivia PNA probe described herein is found in the BV biofilm. G. vaginalis will form the initial layer of the BV biofilm; P. bivia will join the upper layers first, followed by A. vaginae.

Identify Molecular Markers Associated with iBV by Using RNA Sequencing to Analyze the Transcriptome of G. vaginalis, P. Bivia, and A. Vaginae

With the advent of cultivation-independent molecular methods, considerably more constituents of the polymicrobial community in BV have been identified. However, follow-up studies have rarely explored their role in BV pathogenesis. It is hypothesized that BV biofilm maturation depends on specific bacterial interactions and these interactions will be probed in vaginal specimens taken in the 14 days prior to iBV.

### Studies

Due to limitations using in vivo biofilms for molecular studies, in vitro models that mimic in vivo conditions have been optimized. These studies provide proof-of-principle for our the experimental design to identify molecular markers, as environmental conditions are well known to have a strong impact on biofilm formation.

G. vaginalis, P. Bivia, and A. Vaginae can Grow in a Modified Chemically Defined Medium Simulating Vaginal Secretions (mGTS)

To better mimic the vaginal environment in vivo, a modified chemically defined medium (mGTS), containing physiological concentrations of common components of the vaginal environment, namely lysozyme, lactoferrin, and human beta defensin 2, which we have shown induces G. vaginalis biofilm formation, will be used. The 3 selected species can grow under these conditions and can form single-species biofilms. While A. vaginae alone minimally forms a biofilm, when grown together with G. vaginalis, it can become 30% (in vitro) (4) to 40% (in vivo) of the biofilm mass. These data are in accordance with epidemiological studies linking A. vaginae co-colonization with G. vaginalis during BV.

Specific Interactions Among P. bivia, A. Vaginae, and G. vaginalis Induce Expression of Key Genes (Cerca, PI).

In vitro data demonstrated that P. bivia and A. vaginae not only incorporate a G. vaginalis-biofilm but also significantly affect G. vaginalis genes associated with biofilm formation and virulence. Note that these molecular interactions are very specific: While A. vaginae strongly upregulated a type-II glycosyl transferase (HMPREF 0424_0821) thought to be involved in biofilm metabolism, P. bivia up-regulated vaginolysin (vly), but did not induce sialidade (sld). These data demonstrate that species-specific interactions affect BV biofilm formation. Importantly, these preliminary experiments probed known virulence genes of G. vaginalis. The proposed RNA-sequencing experiments will provide a more accurate and unbiased list of molecular markers associated with iBV.

In Vitro Co-Culturing of G. vaginalis, P. Bivia, and A. Vaginae

Multi-species biofilms with 2 or 3 species combinations will be optimized in mGTS in anaerobic conditions (AnaeroPack™, Thermo Fisher Scientific) including key components of the vaginal immune environment. The presence of each species in the dual or tri-species biofilms will be determined by PNA-FISH (as in Aim 2). At least 2 strains per species will be used: one type culture (ATCC 14018, ATCC 29303, and ATCC BAA-55) and at least 1 clinical isolate to assess for possible strain-to-strain variability. If ATCC variants cannot form biofilms, they will be co-cultured planktonically.

### RNA Extraction and Sequencing of the In Vitro Biofilms

RNA will be extracted from dual and tri-species biofilms. RNA quality will be assessed using an Experion automated electrophoresis system (Bio-Rad, Hercules, CA) and samples with an RNA Quality Indicator below 8 will be excluded from the cDNA library. High-quality RNA will be sent for sequencing on dry ice. Library construction and RNA sequencing will be outsourced to EuroFins Genomics (www.eurofinsgenomics.eu), a leading EU-based company, which uses an Illumina MiSeq approach with paired-end reads (2×150 bp).

### Analysis of RNA Sequencing Data

RNA sequencing data will be analyzed following protocols optimized for G. vaginalis (47). CLC Genomics Workbench software will be used for the removal of sequence adapters, mapping to reference genomes, and normalization of gene expression. After alignment of the reference genomes of each species of interest, gene expression will be normalized by calculating reads per kilobase per million mapped reads (RPKM). To assess the consistency of RNA sequencing replicates, RPKM values between replicates will be correlated. Significant gene expression will be determined via Baggerley's test (beta-binominal) with a Bonferroni correction. Venn-diagram analysis will select the most up-regulated genes shared in the in vitro assays.

RNA Extraction and qPCR of In Vivo Biofilms

To validate the potential clinical application of the transcriptomic markers obtained from the in vitro RNA-sequencing data, aliquots of archived vaginal specimens from women with iBV (for the 14 days prior to iBV) and women maintaining normal vaginal microbiota (negative controls) will be shipped from UAB to Minho University on dry ice during Years 3-5 (shipment takes 2 days). RNA extraction, cDNA synthesis, and qPCR quantification (CFX96, Bio-Rad) will be performed as optimized for biofilm cultures, following MIQE guidelines. Housekeeping and specific genes for qPCR (selected from Aim 3A RNA sequencing data) will be designed using Primer3. Primer efficiencies will be optimized using a thermal gradient and the selected melting temperature will include primer sets with equivalent PCR efficiencies (>90%). qPCR specificity will be confirmed by a melting curve at the end of the amplification cycle.

Ideally, RNA sequencing would be performed in vivo however, participant vaginal specimens will likely not meet stringent technical requirements required for this technique. To circumvent this limitation, an optimized in vitro vaginal exudate model (mGTS) will be used. Data obtained from Aim 3A will allow us to obtain a high quality transcriptomic profile that will provide novel data regarding multi-species interactions within BV biofilms. Furthermore, specific genes highlighted by RNA sequencing data will be validated with in vivo specimens in using a less stringent technique (qPCR).

1. A nucleic acid probe comprising a nucleic acid sequence having at least 80% identity to SEQ ID NO: 1 (ATTAAACGTCCATGTCC), wherein the nucleic acid sequence is linked to a detectable moiety.

2. The nucleic acid probe of claim 1, wherein the detectable moiety is a fluorophore.

3. The nucleic acid probe of claim 1, the nucleic acid sequence is a peptide nucleic acid (PNA) sequence.

4. The nucleic acid probe of claim 1, wherein the nucleic acid sequence specifically hybridizes to a Protella bivia nucleic acid.

5. A method for detecting Prevotella bivia in a biological sample from a subject, comprising:
(a) contacting the biological sample with the nucleic acid probe of claim 1; and
(b) detecting the presence of a complex between the nucleic acid probe and a Prevotella bivia nucleic acid in the biological sample.

6. A method of detecting one or more bacteria selected from the group consisting of Prevotella bivia, F. vaginae, and Gardnerella, in a biological sample from a subject, the method comprising:
a. contacting the biological sample with:
i. a first nucleic acid probe comprising a nucleic acid sequence having at least 80% identity to SEQ ID NO: 1; and/or
ii. a second nucleic acid probe comprising SEQ ID NO: 2 (CAGCATTACCACCCG), and/or
iii. a third nucleic acid probe comprising SEQ ID NO: 3 (CGATGTGCGACTAAA);
b. detecting the presence of a complex between the nucleic acid probe comprising SEQ ID NO: 1 and a Prevotella bivia nucleic acid, a complex between the nucleic acid probe comprising SEQ ID NO: 2 and a Gardnerella nucleic acid; and/or a complex between the nucleic acid probe comprising SEQ ID NO: 3 and a F. vaginae nucleic acid.

7. The method of claim 6, wherein the sample is contacted with the first nucleic acid probe comprising a nucleic acid sequence having at least 80% identity to SEQ ID NO: 1.

8. The method of claim 6, wherein the sample is contacted with the first nucleic acid probe comprising a nucleic acid sequence having at least 80% identity to SEQ ID NO: 1; and the second nucleic acid probe comprising SEQ ID NO: 2.

9. The method of claim 6, wherein the sample is contacted with the first nucleic acid probe comprising a nucleic acid sequence having at least 80% identity to SEQ ID NO: 1; and the nucleic acid probe comprising SEQ ID NO: 3.

10. The method of claim 6, wherein the sample is contacted with the first nucleic acid probe comprising a nucleic acid sequence having at least 80% identity to SEQ ID NO: 1; the second nucleic acid probe comprising SEQ ID NO: 2; and the third nucleic acid probe comprising SEQ ID NO: 3.

11. The method of claim 6, wherein the first nucleic acid probe is linked to a first detectable moiety.

12. The method of claim 6, wherein the second nucleic acid probe is linked to a second detectable moiety.

13. The method of claim 6, wherein the third nucleic acid probe is linked to a third detectable moiety.

14. The method of claim 11, wherein each of the first, second and third detectable moieties is a different detectable moiety.

15. The method of claim 14, wherein the detectable moiety is a fluorophore.

16. The method of claim 6, wherein the first, second, and/or third nucleic acid probe is a PNA probe.

17. The method of claim 5, wherein the biological sample comprises cells, tissues or fluid obtained from a patient suspected of having bacterial vaginosis.

18. The method of claim 17, wherein the biological sample comprises cells, tissues or fluid from a vaginal swab.

19. The method of claim 5, wherein detection of the one or more bacteria comprises hybridization.

20. The method of claim 5, wherein the one or more bacteria are detected using fluorescent in situ hybridization (FISH).

21. The method of claim 5, wherein detection of the complex indicates the subject has bacterial vaginosis.

22. A kit comprising:
i. a nucleic acid probe comprising a nucleic acid sequence having at least 80% identity to SEQ ID NO: 1,
ii. a nucleic acid probe comprising a nucleic acid sequence having at least 80% identity to SEQ ID NO: 2 (CAGCATTACCACCCG), and
iii. a nucleic acid probe comprising a nucleic acid sequence having at least 80% identity to SEQ ID NO: 3 (CGATGTGCGACTAAA).

23. The kit of claim 22, wherein the nucleic acid sequences are PNA sequences.


SEQUENCE LISTING (16 sequences)

SEQ ID NO: 1 - DNA, 17 bp, synthetic construct
  source 1..17: mol_type = other DNA
attaaacgtccatgtcc

SEQ ID NO: 2 - DNA, 15 bp, synthetic construct
  source 1..15: mol_type = other DNA
cagcattaccacccg

SEQ ID NO: 3 - DNA, 15 bp, synthetic construct
  source 1..15: mol_type = other DNA
cgatgtgcgactaaa

SEQ ID NO: 4 - DNA, 52 bp, synthetic construct
  source 1..52: mol_type = other DNA
tcgtcggcagcgtcagatgtgtataagagacaggtgccagcmgccgcggtaa

SEQ ID NO: 5 - DNA, 54 bp, synthetic construct
  source 1..54: mol_type = other DNA
gtctcgtgggctcggagatgtgtataagagacagggactachvgggtwtctaat

SEQ ID NO: 6 - DNA, 43 bp, synthetic construct
  source 1..43: mol_type = other DNA
aatgatacggcgaccaccgagatctacactcgtcggcagcgtc

SEQ ID NO: 7 - DNA, 39 bp, synthetic construct
  source 1..39: mol_type = other DNA
caagcagaagacggcatacgagatgtctcgtgggctcgg

SEQ ID NO: 8 - DNA, 20 bp, synthetic construct
  source 1..20: mol_type = other DNA
actcctrcgggaggcagcag

SEQ ID NO: 9 - DNA, 20 bp, synthetic construct
  source 1..20: mol_type = other DNA
ggactaccvgggtatctaat

SEQ ID NO: 10 - DNA, 19 bp, synthetic construct
  source 1..19: mol_type = other DNA
tkaccgcggctgctggcac

SEQ ID NO: 11 - DNA, 21 bp, synthetic construct
  source 1..21: mol_type = other DNA
cacattgggactgagatacgg

SEQ ID NO: 12 - DNA, 20 bp, synthetic construct
  source 1..20: mol_type = other DNA
aggtacactcacccgaaagc

SEQ ID NO: 13 - DNA, 20 bp, synthetic construct
  source 1..20: mol_type = other DNA
cgcacagtaaacgatggatg

SEQ ID NO: 14 - DNA, 20 bp, synthetic construct
  source 1..20: mol_type = other DNA
atgcagcaccttcacagatg

SEQ ID NO: 15 - DNA, 22 bp, synthetic construct
  source 1..22: mol_type = other DNA
tatatcgcatgatgtatatggg

SEQ ID NO: 16 - DNA, 20 bp, synthetic construct
  source 1..20: mol_type = other DNA
catttcaccgctacacttgg